(19) United States
(12) Patent Application Publication
(10) Pub. No.: US 2008/0146856 A1
(43) Pub. Date: Jun. 19, 2008

(54) Propylene production

(76) Inventor: David W. Leyshon, West Chester, PA (US)

Correspondence Address: LyondellBasell Industries, 3801 WEST CHESTER PIKE, NEWTOWN SQUARE, PA, 19073, US

(21) Appl. No.: 11/641,230

(22) Filed: Dec. 19, 2006

(51) Int. Cl. C07C 2/02

(52) U.S. Cl. 585/315

(57) ABSTRACT

A process for producing propylene from ethylene and a feed stream comprising 1-butene is disclosed. The feed stream is contacted with an isomerization catalyst to produce an isomerized stream. The isomerized stream is reacted with ethylene in a distillation column reactor containing a metathesis catalyst to generate a reaction mixture; and the reaction mixture is concurrently distilled to produce an overhead stream comprising ethylene and propylene, and a bottoms stream comprising 1-butene, 2-butene, and C₅ and higher olefins. Propylene is separated from the overhead stream.

### FIELD OF THE INVENTION

The invention relates to a process for producing propylene from ethylene and a feed stream comprising 1-butene.

### BACKGROUND OF THE INVENTION

Steam cracking of hydrocarbons is a petrochemical process that is widely used to produce olefins such as ethylene, propylene, butenes (1-butene, cis- and trans-2-butenes, isobutene), butadiene, and aromatics such as benzene, toluene, and xylene. In an olefin plant, a hydrocarbon feedstock such as naphtha, gas oil, or other fractions of whole crude oil is mixed with steam. This mixture, after preheating, is subjected to severe thermal cracking at elevated temperatures (800° C. to 850° C.) in a pyrolysis furnace. The cracked effluent from the pyrolysis furnace contains gaseous hydrocarbons of great variety (from 1 to 35 carbon atoms per molecule). This effluent contains hydrocarbons that are aliphatic, aromatic, saturated, and unsaturated, and may contain significant amounts of molecular hydrogen. The cracked product of a pyrolysis furnace is then further processed in the olefin plant to produce, as products of the plant, various individual product streams such as hydrogen, ethylene, propylene, mixed hydrocarbons having four or five carbon atoms per molecule (crude C₄'s and C₅'s), and pyrolysis gasoline.

Crude C₄'s can contain varying amounts of n-butane, isobutane, 1-butene, 2-butene (cis- and/or trans-), isobutene(isobutylene), acetylenes(ethyl acetylene and vinyl acetylene), and butadiene. The term 2-butene as used herein includes cis-2-butene, trans-2-butene, or a mixture of both, see N. Calamur, et al., “Butylenes,” in Kirk-Othmer Encyclopedia of Chemical Technology, online edition, 2006.

Crude C₄'s are typically subjected to butadiene extraction or butadiene selective hydrogenation to remove most, if not essentially all, of the butadiene and acetylenes present. Thereafter the C₄ raffinate (called raffinate-1) is subjected to a chemical reaction (e.g., etherification, hydration, dimerization) wherein the isobutylene is converted to other compounds (e.g., methyl tertiary butyl ether, tertiary butyl alcohol, diisobutylene) (see, e.g., U.S. Pat. Nos. 6,586,649 and 4,242,530). The remaining C₄ stream containing mainly n-butane, isobutane, 1-butene and 2-butene is called raffinate-2.

Paraffins (n-butane and isobutane) can be separated from the butenes (1-butene and 2-butene) by extractive distillation. Butenes can be reacted with ethylene to produce propylene through isomerization and metathesis reactions (Appl. Ind. Catal. 3 (1984)215).

Streams containing 1-butene are available from other petrochemical processes as well. For example, such a stream may be a condensate derived from a Fisher-Tropsch process by reacting a synthesis gas mixture including carbon monoxide and hydrogen over a Fisher-Tropsch catalyst (see, Catal. Lett. 7(1-4) (1990)317).

Catalytic distillation of various hydrocarbon streams for various purposes such as hydrogenation, olefin isomerization, etherification, dimerization, hydration, and aromatic alkylation, and metathesis has been disclosed, see U.S. Pat. Nos. 4,443,559, 6,495,732 4,935,577, 6,583,329, 6,515,193, 6,518,469, U.S. Pat. Appl. Pub. Nos. 2004/0192994, 2006/0089517, and Chem. Eng. Prog. March 1992, 43.

### SUMMARY OF THE INVENTION

The invention is a process for producing propylene. A feed stream comprising 1-butene is contacted with an isomerization catalyst to obtain an isomerized stream comprising 2-butene. The isomerized stream is reacted with ethylene in a distillation column reactor containing a metathesis catalyst to produce a reaction mixture comprising ethylene, propylene, 1-butene, 2-butene, and C₅ and higher olefins. Concurrently the reaction mixture is distilled to produce an overhead stream comprising ethylene and propylene, and a bottoms stream comprising 1-butene, 2-butene, and C₅ and higher olefins. Propylene is separated from the overhead stream.

### BRIEF DESCRIPTION OF THE DRAWING

FIG. 1 is schematic flow diagram of one embodiment of the invention.

### DETAILED DESCRIPTION OF THE INVENTION

The process of the invention comprises: (a) contacting a feed stream comprising 1-butene with an isomerization catalyst to produce an isomerized stream; (b) reacting the isomerized stream and ethylene in a distillation column reactor containing a metathesis catalyst to generate a reaction mixture, and concurrently distilling the reaction mixture to produce an overhead stream comprising ethylene and propylene, and a bottoms stream comprising 1-butene, 2-butene, and C₅ and higher olefins (olefins containing five or more carbons); and (c) separating the overhead stream into propylene and ethylene.

The feed stream for this invention may be any suitable stream that comprises 1-butene. The feed stream may comprise other components such as ethylene, propylene, 2-butene, n-butane, isobutene, butadiene, acetylenes, C₅ and higher hydrocarbons (hydrocarbon molecules containing five or more carbon atoms). One suitable feed stream is called raffinate-2, which is obtained from a crude C₄ stream from refining or steam cracking processes. Raffinate-2 contains mostly 1-butene, 2-butene, n-butane, and isobutane. Preferably, paraffins (n-butane and isobutane) are removed from raffinate-2 by extractive distillation with a suitable extractive solvent (e.g., dimethyl formamide, N-methyl pyrrollidone, or N-formyl morpholine) or selective adsorption. These techniques are described in U.S. Pat. Nos. 4,515,661, 5,288,370, U.S. Pat. Appl. Pub. No. 2005/0154246, and DeRosset, A. J., et al., Prepr.—Am. Chem. Soc., Div. Pet. Chem. (1978)23(2) 766. See also “Technology Profile Butenex®,” available is from company web http://www.uhde.biz/company/index.en.epl. Another suitable feed stream is a condensate from a Fisher-Tropsch process obtained by reacting a synthesis gas mixture including carbon monoxide and hydrogen over a Fisher-Tropsch catalyst (Catal. Lett. 7(1-4) (1990)317). The condensate typically contains ethylene, propylene, C₄ olefins, and C₅ and higher olefins. When a Fischer-Tropsch-derived feed as described above is used, it may be optionally fractionated to remove C₅ and higher hydrocarbons by distillation or other methods (see, e.g., U.S. Pat. No. 6,586,649).

Preferably, the feed stream is primarily composed of 1-butene and 2-butene. For example, the amount of 1-butene and 2-butene combined in the feed stream is desirably at least 95 weight percent (wt. %), more desirably at least 99 wt. %. The relative amount of 1-butene and 2-butene in the feed is not critical.

The feed stream is contacted with an isomerization catalyst to produce an isomerized stream. At least a portion of 1-butene in the feed stream is converted to 2-butene by the isomerization. The relative molar ratio of 1-butene to 2-butene in the isomerized stream is preferably in the range of 9:1 to 1:9. More preferably, the ratio is in the range of 1:1 to 1:5.

Many isomerization catalysts can be used, including acidic catalysts, basic catalysts, and hydroisomerization catalysts. Suitable acidic catalysts include acidic ion-exchange resins such as sulfonated resins (see, e.g., U.S. Pat. No. 3,326,866), organosulfonic acids, phosphoric acid, carboxylic acids, metal oxides (alumina, zirconia, sulfated zirconia), mixed oxides (e.g., silica-alumina, zirconia-silica), acidic zeolites, acidic clays (see, e.g., U.S. Pat. No. 4,992,613, U.S. Pat. Appl. Pub. Nos. 2004/249229, 2006/084831). Acidic ion-exchange resins are preferred.

When an acidic catalyst is used, the isomerization is typically conducted at a temperature from 40 to 200° C., preferably from 90 to 150° C., and under a pressure of 700 to 2800 kPa (10³ Pascal), preferably from 1,000 to 1,500 kPa. The weight hourly space velocities, WHSV, are generally maintained at 0.2 to 4 kg feed per kg catalyst per hour.

The basic isomerization catalysts are preferably metal oxides such as magnesium oxide (magnesia), calcium oxide, barium oxide, and lithium oxide. Metal oxides supported on a carrier may be used. Suitable carriers include silica, alumina, titania, silica/alumina, and the like, and mixtures thereof (see, e.g., U.S. Pat. Nos. 5,153,165, 5,300,718, 5,120,894, 4,992,612, U.S. Pat. Appl. Pub. No. 2003/0004385). A particularly preferred basic isomerization catalyst is magnesium oxide. Suitable magnesium oxide has a surface area of at least 1 m²/g, preferably >5 m²/g. The magnesium oxide is preferably activated in a suitable manner, for example, by heating in a flowing stream of an oxygen-containing gas for about 1 to about 30 hours at 250 to 800° C., preferably at 300 to 600° C. before use.

Isomerization in the presence of magnesium oxide catalyst may be conducted at a temperature ranging from 50 to 500° C., preferably ranging from 150 to 450° C., most preferably ranging from 250 to 300° C., and at a pressure and a residence time effective to give a desired composition of the isomerized stream.

The isomerization may be catalyzed by a hydroisomerization catalyst in the presence of small amount of hydrogen. Hydroisomerization reaction of olefins is well known (Hydrocarbon Process., Int. Ed. May 1979, 112). Suitable catalysts include supported noble metal catalysts (e.g., Pd or Pt supported on silica or alumina, see U.S. Pat. No. 3,531,545). The hydrogen to hydrocarbon feed molar ratio is typically in the range of 1:10 to 1:100. The hydroisomerization is usually conducted at a temperature of 30 to 150° C., preferably 40 to 100° C., and under a pressure of 700 to 3,000 kPa, preferably from 1,000 to 1,500 kPa. The weight hourly space velocity, WHSV, may be maintained at 0.1 to 20, preferably 1 to 10 kg feed per kg catalyst per hour.

The hydroisomerization of the feed stream is particularly preferred if the feed stream contains small amount of butadiene or acetylenes. A hydroisomerization process not only converts 1-butene to 2-butene, it also converts butadiene or C₄-acetylenes to mono-olefins such as 1-butene and 2-butene.

The isomerization catalysts are preferably beads, granules, pellets, extrudates, tablets, agglomerates, and the like. The catalyst is preferably used in a fixed bed and the reaction is performed in a continuous flow mode.

The isomerized stream is reacted with ethylene in a distillation column reactor containing a metathesis catalyst to form a reaction mixture comprising ethylene, propylene, 1-butene, 2-butene, and C₅ and higher olefins. In a distillation column reactor, reactants are converted to products over a catalyst and at the same time distillation of the reaction mixture occurs to separate the mixture into two or more fractions. Such a technique is called reactive distillation or catalytic distillation. Catalytic distillation is well known in chemical and petrochemical industries (see, e.g., U.S. Pat. Nos. 4,935,577, 5,395,981, 5,196,612, 5,744,645, U.S. Pat. Appl. Pub. Nos. 2004/0192994, 2005/080309, and 2006/052652). Olefin metathesis is known to be carried out in a distillation column reactor (see, e.g., U.S. Pat. Nos. 6,583,329, 6,515,193, and 6,518,469).

A metathesis catalyst is contained in the distillation column reactor. Metathesis catalysts are well known in the art (see, e.g., Appl. Ind. Catal. 3 (1984)215). Typically, the metathesis catalyst comprises a transition metal oxide. Suitable transition metal oxides include oxides of cobalt, molybdenum, rhenium, tungsten, and mixtures thereof. Conveniently, the catalyst is supported on a carrier. Suitable carriers include silica, alumina, titania, zirconia, zeolites, clays, and mixtures thereof. Silica and alumina are preferred. The catalyst may be supported on a carrier in any convenient fashion, in particular by adsorption, ion-exchange, impregnation, or sublimation. The transition metal oxide constituent of the catalyst may amount to 1 to 30 wt. % of the total catalyst, preferably 5 to 20 wt. %.

A catalyst comprising rhenium oxide supported on alumina is active at relatively low temperature (<100° C.) and is particularly suitable for the present invention. Such catalyst may be prepared by impregnating a high-surface-area alumina with an aqueous ammonium perrhenate solution (Appl. Ind. Catal. 3 (1984)215).

In the distillation column reactor, the metathesis catalyst functions both as a catalyst and as distillation packings. In other words, packings in a column distillation reactor serve both a distillation function and a catalytic function.

The metathesis catalyst may be a powder or particulates. Particulate metathesis catalysts are preferred. The catalyst particles such as beads, granules, pellets, extrudates, tablets, agglomerates, honeycomb monolith, and the like must be sufficiently large so as not to cause high pressure drops through the column. Alternatively, the catalyst may be incorporated into the packings or other structures (see Chem. Eng. Prog. March 1992, 43). Preferred catalyst structure for use in the distillation column reactors comprises flexible, semi-rigid open mesh tubular material, such as stainless steel wire mesh, filled with a particulate metathesis catalyst. Other structures suitable for the present invention can be found in U.S. Pat. Nos. 4,242,530, 4,443,559. 4,536,373, 4,731,229, 4,774,364, 4,847,430, 5,073,236, 5,348,710, 5,431,890, and 5,510,089. For example, U.S. Pat. Nos. 4,242,530 and 4,443,559 disclose particulate catalysts in a plurality of pockets in a cloth belt or wire mesh tubular structures, which are supported in the distillation column reactor by open mesh knitted stainless steel wire by twisting the two together into a helix.

Optionally, additional internal stages in the form of packings or trays are installed above and/or below the catalyst bed. Preferably, a stripping section is below the catalyst bed and a rectification section is above the bed. The distillation column reactor is typically equipped with an overhead cooler, condenser, a reflux pump, a reboiler, and standard control instrumentations.

The distillation column reactor will contain a vapor phase and a liquid phase, as in any distillation. The success of the concurrent distillation and reaction approach lies in an understanding of the principles associated with distillation. First, because the reaction is occurring concurrently with distillation, the initial reaction products are removed from the reaction zone as quickly as possible. Second, because all the components are boiling, the reaction temperature is controlled by the boiling point of the mixture at the system pressure. The heat of reaction simply creates more boiling, but no increase in temperature. Third, the reaction has an increased driving force because the reaction products are removed and cannot contribute to a reverse reaction. As a result, a great deal of control over the rate of reaction and distribution of products can be achieved by regulating the system pressure.

The temperature in a distillation column reactor is determined by the boiling point of the liquid mixture present at a given pressure. The temperature in the lower portions of the column will reflect the composition of the material in that part of the column, which will be higher than the overhead; that is, at constant pressure a change in the temperature of the system indicates a change in the composition in the column. Temperature control in the reaction zone is thus effected by a change in pressure; by increasing the pressure, the temperature in the system is increased, and vice versa. The pressure of the distillation column reactor is high enough to condense 1-butene, 2-butene, and C₅ and higher olefins but low enough to allow ethylene and propylene to exit the partial condenser as vapor and to reduce the propylene concentration in the catalyst pores, thus shifting equilibrium toward propylene. Suitable temperatures to operate this column are in the range of 40 to 150° C., and the pressure ranges from 1,500 to 3,500 kPa.

The isomerized stream is preferably fed above the catalyst bed and the ethylene is preferably fed as gas below the catalyst bed. The ethylene flows upward into the catalyst bed and reacts to form propylene which is removed as an overhead stream along with small amount of non-reacted ethylene. In a distillation column reactor, the equilibrium is constantly disturbed, thus although the equilibrium concentration of propylene at a given temperature is rather low, the removal of the propylene as an overhead product constantly drives the reaction to produce propylene. C₅ and higher olefins (e.g., pentenes, hexenes) may be produced as a result of the metathesis reaction of 2-butene and 1-butene. Another advantage of the catalytic distillation reactor is that the feeds to the metathesis reactor are dried by azeotropic distillation allowing long periods of catalytic activity without the special drying steps that would otherwise be necessary. The necessity for dry feed is indicated in U.S. Pat. No. 3,340,322.

The rectification section above the bed, if used, ensures that butenes (1-butene and 2-butene) and C₅ and higher olefins are separated from the propylene product and non-reacted ethylene. The bottoms stream is taken to remove 1-butene, 2-butene, and C₅ and higher olefins present in the reactor. A mixture of primarily ethylene and propylene is taken as an overhead stream from the column reactor.

Propylene is separated from the overhead stream using standard techniques. For example, propylene and ethylene can be separated by fractional distillation, which is well known in the art. The distillation may be operated at a temperature in the range of −20 to 100° C., and a pressure in the range of 3,000 to 4,500 kPa. The separated ethylene stream may be recycled to the distillation column reactor of step (b).

The bottoms stream comprising 1-butene, 2-butene, and C₅ and higher olefins may be distilled to separate 1-butene and 2-butene as a light stream, while the C₅ and higher olefins is taken as a heavy stream. The light stream may be recycled to the isomerization step. The distillation column for this separation may be equipped with any packing which is effective for the desired separation. The distillation may be operated at a temperature in the range of 50 to 175° C. and a pressure of 1,000 to 2,000 kPa.

### EXAMPLE

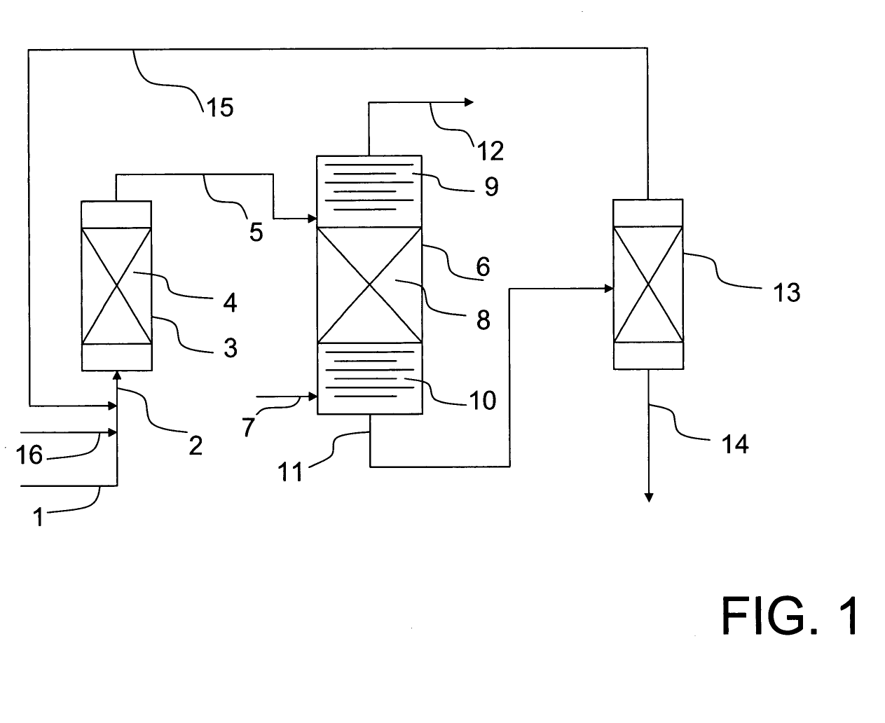

The following example is based on ASPEN simulations of a scheme shown in FIG. 1. A feed stream is produced by removing C₄ paraffins from a raffinate-2 stream by extractive distillation. The expected composition of the feed stream is shown in Table 1. The feed stream via line 1, hydrogen via line 16, and the recycled C₄ stream via line 15 are mixed and the mixture is fed to the isomerization reactor 3 via line 2. The isomerization reactor 3 contains a Pd/alumina (0.3 wt % Pd) catalyst bed 4. The isomerization reaction is conducted at 80° C. and 1200 kPa. The WHSV of this reaction is 4 kg of C₄ hydrocarbons per kg of catalyst per hour.

The effluent from the isomerization reactor (called isomerized stream) is fed to the catalytic distillation reactor 6 via line 5. The catalytic distillation reactor 6 consists of a catalyst bed 8 (15 stages) containing a Re/alumina catalyst (7 wt. % Re, particle size 3/16 inch), a rectifying section 9 having 8 ideal stages above the bed, and a stripping section 10 having 7 stages below the catalyst bed. The isomerized stream is fed at the 7th stage of the reactor. Ethylene enters the tower via line 7 and passes through the stripping section 10 and the catalyst bed 8 to produce propylene via metathesis reaction with 2-butene. The temperatures are maintained at about 48° C. (top) to 132° C. (bottom) at an operating pressure of 2,800 kPa within the tower. Unconverted ethylene and the formed propylene exit the tower via line 12. The ethylene and propylene are separated elsewhere.

Unconverted butenes (1-butene and 2-butene), C₅ and C₆ olefins, and possibly other heavier olefins exit the bottom of the reactor tower via line 11 and are fed to tower 13 which separates the C₅ and higher olefins stream from the butenes recycle stream. The tower 13 has 16 ideal stages and is operated at a temperature range of 92° C. (top) to 140° C. (bottom) and at a pressure of 1,400 kPa. The butene recycle stream is fed back to line 2 via line 15. The C₅ and higher olefins stream exits tower 13 via line 14. The calculated flow rates of different components in various lines are listed in Table 1.

TABLE 1
Material Balance (kg/h)

| | Line # | | | | | | |
|---|---|---|---|---|---|---|---|
| | 16 | 1 | 2 | 11 | 12 | 14 | 7 |
| Hydrogen | 125 | | 125 | | 100 | | |
| Ethylene | | | 2 | 2 | 29196 | | 76223 |
| Propylene | | | 1456 | 1456 | 143855 | | |
| 2-Butene | | 38500 | 90267 | 51767 | 1221 | | |
| 1-Butene | | 61488 | 63911 | 2423 | 238 | | |
| C5 Olefins | | | | 1557 | | 1557 | |
| C6 Olefins | | | | 144 | | 144 | |
| Total | 125 | 99988 | 155761 | 57349 | 174610 | 1701 | 76223 |

I claim:

1. A process for producing propylene comprising:
(a) contacting a feed stream comprising 1-butene with an isomerization catalyst to obtain an isomerized stream comprising 2-butene;
(b) reacting the isomerized stream and ethylene in a distillation column reactor containing a metathesis catalyst to generate a reaction mixture, and concurrently distilling the reaction mixture to produce an overhead stream comprising ethylene and propylene, and a bottoms stream comprising 1-butene, 2-butene, and C₅ and higher olefins; and
(c) separating the overhead stream into propylene and ethylene.

2. The process of claim 1 further comprising distilling the bottoms stream to obtain a light stream comprising 1-butene and 2-butene, and a heavy stream comprising the C₅ and higher olefins.

3. The process of claim 2 wherein the bottoms stream is distilled at a temperature in the range of 50 to 170° C..

4. The process of claim 2 wherein the bottoms stream is distilled at a pressure in the range of 1,000 to 2,000 kPa.

5. The process of claim 2 further comprising recycling the light stream to step (a).

6. The process of claim 1 further comprising recycling the separated ethylene to step (b).

7. The process of claim 1 wherein the isomerization catalyst is an acidic catalyst.

8. The process of claim 1 wherein the isomerization catalyst is an acidic ion-exchange resin.

9. The process of claim 1 wherein the isomerization catalyst is a basic catalyst.

10. The process of claim 1 wherein the isomerization catalyst comprises magnesium oxide.

11. The process of claim 1 wherein the isomerization catalyst is a hydroisomerization catalyst.

12. The process of claim 11 wherein the hydroisomerization catalyst comprises Pd and alumina.

13. The process of claim 1 wherein the metathesis catalyst comprises a transition metal oxide comprising an element selected from the group consisting of cobalt, molybdenum, rhenium, tungsten, and mixtures thereof.

14. The process of claim 1 wherein the metathesis catalyst comprises rhenium oxide and alumina.

15. The process of claim 1 wherein the step (b) is conducted at a temperature in the range of 40 to 150° C..

16. The process of claim 1 wherein the step (b) is conducted at a pressure in the range of 1,500 to 3,500 kPa.

17. The process of claim 1 wherein the isomerized stream is fed to the distillation column reactor above the catalyst bed.

18. The process of claim 1 wherein the ethylene is fed to the distillation column reactor below the catalyst bed.

19. The process of claim 1 wherein step (c) is performed by distillation at a temperature in the range of −20 to 100° C. and a pressure in the range of 3,000 to 4,500 kPa.
